(19) United States
(12) Patent Application Publication
(10) Pub. No.: US 2012/0203182 A1
(43) Pub. Date: Aug. 9, 2012

(54) Adhesive layer arrangements and methods for securing medical tubing

(76) Inventors: Dennis M. Kay, Tampa, FL (US); Steven J. Babb, Seminole, FL (US); David W. Underwood, Tampa, FL (US); John A. DeBella, Clearwater, FL (US); Darrell J. Carrens, Tampa, FL (US)

(21) Appl. No.: 13/136,669

(22) Filed: Aug. 5, 2011

Related U.S. Application Data
(63) Continuation-in-part of application No. 12/620,844, filed on Nov. 18, 2009.
(60) Provisional application No. 61/401,036, filed on Aug. 6, 2010.

(51) Int. Cl. A61M 25/02

(52) U.S. Cl. 604/180

(57) ABSTRACT

Adhesive layer arrangements for securing catheters relative to a patient's body and forming occlusive seals at catheter insertion sites.

### CROSS-REFERENCE TO RELATED PATENT APPLICATION

The present application is a continuation-in-part of U.S. patent application Ser. No. 12/620,844 filed on Nov. 18, 2009, and claims priority from provisional patent application Ser. No. 61/401,036 filed on Aug. 6, 2011, the entire disclosures of which are incorporated herein by reference.

### BACKGROUND OF THE INVENTION

1. Field of the Invention

The present invention pertains to adhesive layer arrangements and devices for securing medical tubing, such as catheters, to a patient's body and methods therefor.

2. Brief Discussion of the Related Art

Medical catheters are used to transfer liquids and gases (fluids) to and/or from naturally or surgically created openings in a patient's body, such as stoma, the oral cavity, the urethra or percutaneous sites such as for venous catheters. Catheter safety and reliability depend on assuring patency of the lumen of the catheter and securement such that physical forces are not transmitted internally to the patient and the exact physical location of the catheter relative to the body is maintained. Design and materials of catheter securement arrangements are needed so as to not cause injury to the patient, such as maceration, pressure ulcers, skin tears and infections for example.

Catheters commonly range in size from 1.9 French (approximately 0.627 mm) for neonatal applications through thoracostomy tubes up to 44 French (approximately 14.52 mm) and include many varieties of mid-size catheters (for example, intravenous, suprapubic, urinary drainage, gastrostomy [PEG], etc.). To reduce caregiver training costs and improve catheter securement and insertion site dressing technique compliance, a safe and reliable catheter securement arrangement is desirable which can be used for the entire range of commonly used catheters.

One of the disadvantages of prior art catheter securement arrangements is that such catheter securement arrangements permit in-and-out motion or “pistoning” of catheters at the point of entry (insertion site) into a patient's body, and even slight in-and-out motion or “pistoning” can cause infection in that the sliding movement of a catheter with respect to an insertion site carries organisms (pathogens) through the epidermal barrier to cause such infections. Such sliding movement is not adequately prevented by medical tape and prior art catheter holders or securement arrangements and devices currently available.

Other disadvantages of the prior art is that catheters are not secured and do not form an occlusive seal using a single arrangement or device, prior art catheter securement arrangements do not remain adhered in the presence of moisture, blood, mucus and other bodily fluids, prior art catheter securement arrangements can cause injury from hard plastic parts for catheter securement. When patients lay atop the securement devices, the hard part can cause wounds or pressure injuries and discomfort. Prior art catheter securement arrangements require specialized securement fixtures for use with multiple types of catheters increasing inventory requirements. Where circumferential elastic bands are used to secure a securement device to the body, injury can be caused by constriction. Contact dermatitis can be caused by acrylic adhesive of prior art securement devices, and dermal injury can be caused when acrylic adhesive is pulled from a patient.

The walls of catheters are typically subject to various physical forces which are potentially detrimental to the patency of the catheter lumen. These forces can be transmitted through the catheter wall and may cause tissue injury to a patient. Polymeric materials used for catheter construction typically exhibit a low flexural modulus, softness (i.e. low durometer) and high modulus of elasticity resulting in catheters which are pliable but resist stretch. Conventional polymeric materials include silicone rubber and various thermoplastic elastomers such as polyisobutylene (latex), polyvinylchloride (PVC), fluorinated ethylene proprylene (FEP), polytetrafluoroethylene (PTFE) and polyurethane. Material properties which make catheters pliable also render the catheters susceptible to crushing such as at rigid pinch points in various prior art catheter securement arrangements. Kinking is another factor which can cause significant loss of catheter patency such as when a catheter is forced to bend excessively over a short distance. Catheters are also subject to twisting forces along the longitudinal axes thereof caused by the arrangement of other medical devices or patient movement. Catheters are also subject to tugging and leverage forces caused by various factors such as bending at the catheter insertion site into a body. Prior art catheter securement arrangements do not adequately protect against the above-mentioned disadvantages such that tissue damage can occur wherever a patient's skin is crushed (ischemia) or torn and also do not permit a controlled and stable angle of entry at the insertion site for a catheter to avoid leverage forces being transmitted through the catheter thereby causing crushing at the inboard interface and tearing at the outboard interface. Detrimental leverage forces also occur with the use of catheter securement devices having a swiveling securement mechanism gripping design.

### SUMMARY OF THE INVENTION

A primary aspect of the present invention is to provide adhesive layer arrangements for securing medical tubing to a body by positively gripping the tubing to avoid the above-described disadvantages of the prior art while facilitating application of the adhesive layer arrangements.

Another aspect of the present invention is to utilize a hydrocolloid layer delivery system to secure medical tubing to the body with a plow-fold release film facilitating adherence of a hydrocolloid layer to skin by originally pulling part of the release film to secure the hydrocolloid layer to the skin and thereafter using the plow-fold configuration to smoothly secure the hydrocolloid layer to the skin. Another release film can be supplied on the opposite side of the hydrocolloid layer such that when the other release layer is removed, medical tubing can be adhesively secured to the hydrocolloid layer. A second hydrocolloid layer can be disposed over the first hydrocolloid layer to hold the medical tubing therebetween, thus preventing lateral, longitudinal and rotational movement of the medical tubing as well as preventing the medical tubing from moving away from the body, i.e. peeling movement.

In a further aspect, an adhesive layer arrangement according to the subject invention provides circumferential gripping of a catheter thereby preventing kinking or collapse of the tubing lumen and resultant loss of catheter patency.

In a another aspect, an adhesive layer arrangement according to the present invention includes a pair of oval-shaped hydrocolloid layers utilizing a plow-fold release layer delivery system to enhance application to skin and limit movement of medical tubing held therebetween, thus forming a hydrocolloid sandwich when upper and lower surfaces of the hydrocolloid layers come together to comingle via cold-flow forming a full 360° seal of hydrocolloid around the medical tubing.

In another aspect, an adhesive layer arrangement according to the present invention produces a transcutaneous seal via a first hydrocolloid layer that can seal around any percutaneous catheter and can be combined with another layer of hydrocolloid to produce a bilaminar sealing mechanism. The arrangement can include a longer strip of hydrocolloid connected with a shorter strip of hydrocolloid by a connecting portion. This 2-dimensional shape can form a 3-dimensional cone which auto-adapts to the size of tubing to be secured. The “tail” or shortest strip of hydrocolloid seals to the skin or to another layer of hydrocolloid and pre-positions the seal in a correct location prior to wrapping of the medical tubing to ensure an occlusive seal around the tubing (catheter) and the transcutaneous tubing insertion site. The “fuselage” or connecting portion forms a seal behind and beneath the medical tubing (i.e. the area between the medical tubing and the skin as it lays parallel to the skin) in a manner that cannot be accomplished by tape. Attachment of the tail section to the skin allows the seal to partially form by simply laying the catheter on top of the seal. The “fuselage” is then exposed as release paper is pulled off the “wings”, and the fuselage sticks to the backside of the catheter and to the wings as the wings fold over to form the seal. The wings fold around the catheter onto the fuselage and the catheter, cross and seal to each other and to the mounting surface such as the skin or hydrocolloid.

Adhesive layer arrangements for securing catheters in accordance with the present invention permit securing a catheter and sealing the percutaneous insertion site with an occlusive seal utilizing a single arrangement/device, the adhesive layer arrangement remains adhered to the body in the presence of moisture and body fluids, the adhesive layer arrangement of the subject invention moves like a patient's skin due to the hydrocolloid and polymer film laminate combination having the same dynamic physical properties as skin, the adhesive layer arrangement can be secured to all types of tubing materials through cold-flow.

Some of the advantages of the present invention over prior art arrangements for securing medical tubing include securement of medical tubing in a simple manner, increased infection control, cooperation with physical properties and physiology of skin to reduce injuries, safety in use due to elimination of structures which could cause tissue cuts, maceration or necrosis, easier training of caregivers and use by caregivers, protection of lumen patency across a wide range of catheter dimensions, materials and clinical applications, reduction of the potential for infection due to the ability to create an occlusive seal against pathogens at catheter insertion sites and/or prevention of catheter dislodgement.

Aspects and features of the present invention include an adhesive layer/laminate with a thickness of less than 30 millimeters thickness, any adhesive materials or laminate than can simulate the same properties as skin in both flexural modulus and elasticity modulus, an adhesive material capable of sealing to skin for more than 3 days, an adhesive material capable of managing moisture transmission from skin without causing over hydration, maceration or wounding (capable of managing moisture output of normal skin and the skin of diaphoretic patients without causing skin breakdown), adhesive layers sized to effectively secure catheters of sizes 1.7 french to 47 french, tabs of the release liner contiguous with the adhesive layer and extending at least to the distal end of the adhesive layer, three separate release film components, an ovoid slot design for mounting the proximal portion of the slot in close approximation with a catheter insertion site, either or both hydrocolloid layers having a window of transparent film (with a border of adhesive/hydrocolloid for adhesion) enhancing insertion site observation, a percutaneous seal may be a single layer used to perform all four necessary functions of adhering to skin, adhering to catheter, forming an occlusive seal around the catheter and forming an occlusive seal over the insertion site, a percutaneous seal may be a bi/multiple layer configuration to perform all four necessary functions of adhering to skin, adhering to catheter, forming an occlusive seal around the catheter, and forming an occlusive seal over the insertion site, a multiple plow-fold release liner/film component assembly can be used to deliver the bottom adhesive layer of the “airplane” device to the desired application site including the skin surface application and an “X” application pattern to create an occlusive seal at the catheter insertion site, a lower adhesive layer may have a top film which is coated with a release agent allowing the removal of the top film and exposing the hydrocolloid to create an adhesive bed for the catheter, a transcutaneous seal release layer extending at least to the periphery of the adhesive layer enabling optimal orientation and application of the seal, and/or a slotted adhesive base for establishing skin contact around a catheter insertion site with the base having at least three separate release film components.

Other aspects and advantages of the present invention will become apparent from the following description of the invention taken in conjunction with the accompanying drawings, wherein like parts in each of the several figures are identified by the same reference characters.

### BRIEF DESCRIPTION OF THE DRAWINGS

FIG. 1 is a perspective view of an adhesive layer arrangement according to the present invention having two layers of hydrocolloid sandwiching a catheter.

FIG. 1A is a perspective view of the adhesive layer arrangement of FIG. 1 without the catheter and with overlapping release films on each layer.

FIG. 2 is a plan view of another embodiment of an adhesive layer arrangement according to the present invention providing an occlusive seal at an insertion site.

FIG. 3 is a plan view of the embodiment of FIG. 2 applied to a catheter to produce an occlusive seal.

FIG. 4 is a broken perspective showing the use of an adhesive layer arrangement along with a catheter.

FIG. 5 is a perspective view showing an adhesive layer arrangement according to the present invention adjacent a catheter to be secured and occlusively sealed relative to an insertion site.

FIG. 5A is a perspective view of an adhesive layer arrangement similar to FIG. 5 without a catheter.

FIG. 6 is a perspective view of the adhesive layer arrangement shown in FIG. 5 including plow-fold release films/layers.

FIGS. 7, 8, 9 and 10 are perspective drawings showing the method of use of the adhesive layer arrangement of FIGS. 5 and 6.

FIG. 11 is an exploded view of another adhesive layer arrangement according to the present invention.

FIGS. 12 and 13 show the method of applying adhesive layer arrangements according to the present invention with captions explaining the steps.

FIGS. 14-29 show method steps for using the adhesive layer arrangement of FIGS. 5, 6 and 10 and variations thereof for securing a catheter to the body and creating an occlusive seal at the insertion site.

### DETAILED DESCRIPTION OF THE INVENTION

As used herein, the term “catheter” means any medical tubing for disposition in or along the body of a patient.

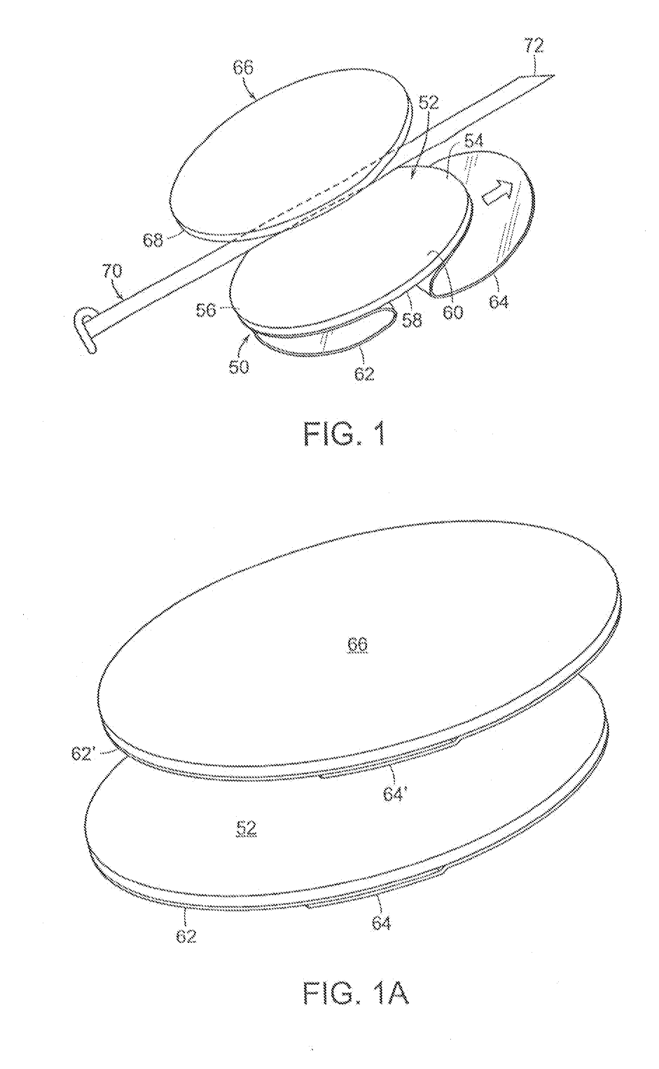

An adhesive layer arrangement 50 according to the present invention for securing a catheter to the body of a patient is shown in FIG. 1 and includes a first or lower layer of hydrocolloid 52 having opposing ends 54 and 56, a bottom adherent surface 58 for adhering to the body of a patient and a top adherent surface 60 that is normally covered by a release film 62′. A plow-fold (U-shaped) release film 62 (shown as two parts) covers the bottom adherent surface 58 and has a tab(s) 64 extending away from the bottom adherent surface.

A second or upper layer of hydrocolloid 66 has a bottom adherent surface 68 for adhering to the top surface 60 of hydrocolloid layer 52 and to a catheter 70 normally inserted into the body of a patient at an insertion site 72. When the upper layer of hydrocolloid 66 is pressed against the lower layer of hydrocolloid 52 with the catheter 70 disposed on the top surface of the layer of hydrocolloid 52, the upper layer of hydrocolloid deforms to securely grip the catheter and, in combination with the hydrocolloid layer 52, secures the catheter to the patient's body preventing rotational and longitudinal movement of the catheter, the hydrocolloid layers producing cold flow of the hydrocolloid.

The release films can be initially applied to the adherent surfaces of the layers 52 and 66 such that the innermost edges of the release films abut one another or, as shown in FIG. 1A, the release films can overlap one another at the tabs 64.

A unique property of hydrocolloids and certain other similar adhesive compositions is the ability to “cold-flow”, filling gaps between adjacent adhesive surfaces or between an adhesive surface and a non-adhesive surface by flowing into areas of adhesive gaps or surface voids in the manner of a supercooled liquid.

The hydrocolloid layers 52 and 66 preferably have oval shapes and are formed as separate layers of hydrocolloid although it is understood that the hydrocolloid layers could be integrally formed. The layers of hydrocolloid produce a low profile sandwich-type catheter securement. As illustrated in FIG. 1, the plow-fold release film facilitates application of the hydrocolloid layers in use. The lower hydrocolloid layer can be extruded onto a removable release film whereby a complete hydrocolloid sandwich is created when the upper and lower hydrocolloid layers come together to comingle via cold-flow phenomenon forming a full 360° of hydrocolloid seal around the catheter.

The adhesive layer arrangement shown in FIG. 1 is based on the use of two separate layers, preferably having oval shapes, either similar or dissimilar in size to form a sandwich of hydrocolloid with the catheter between the layers. The oval sandwich can have the bottom oval carrying a polyurethane film presenting to the catheter or have a release film top layer which, when removed, presents an adherent surface of the layer of hydrocolloid to the catheter and also to the adherent bottom surface of the upper hydrocolloid layer that also presents hydrocolloid to the catheter as well. If the polyurethane film is in place, the opposing hydrocolloid of the top oval will adhere to the outer, upper two-thirds, of the circumference of the catheter for securement and hold the catheter in place while also providing a film on which to mount a device or adhere to any tape used as well. Where the release film option is used, the lower hydrocolloid layer adheres to the opposing exposed lower surface of the upper hydrocolloid layer to create a full adhesive seal around the catheter completely sandwiched by a fully circumferential mass of hydrocolloid.

A tube holder such as disclosed in U.S. patent application Ser. No. 12/620,844, or any other tube fixation device, can be mounted on the lower hydrocolloid layer. Where a tube holder is used, the lower hydrocolloid layer preferably has a slot in one end forming an opening to receive a catheter with a tube holder mounted on the opposite end of the lower hydrocolloid layer as described and shown hereinafter.

Additionally, an equally slotted oval of the same or differing size, as shown in FIG. 11, can be used. The oval shape has radiused ends to resist roll-up and premature release. The sandwich presents a low profile when no tube holder is utilized; and, in many instances, no mechanical fixation, such as via a tube holder, is required due to the strength of the adhering properties of the two layers of hydrocolloid. Additionally, the two layers of hydrocolloid prevent kinking of catheters and replaces the need for tape on tape-allergic patients or tape-sensitive patients (neonatal). Once applied, neither skin moisture, perspiration or other body fluids or external moisture can cause premature, quick release of skin attachment as can occur with acrylic-based adhesives of the prior art. The hydrocolloid layers can adhere to all tubing materials, such as silicone, polyurethane and nylon and prevent catheter “wander” or drift by ensuring the catheter is fixed to the skin. Additionally, the use of the two hydrocolloid layers prevents catheter “pistoning” in and out of insertion sites, reducing phlebitis. The entire sandwich of hydrocolloid layers with or without polyurethane film will mimic the properties of intact skin in that the modulus of elasticity is similar to intact skin. When the two hydrocolloid layer arrangement is applied to thin, compromised, “at risk” or “rice paper” type skin, the potential for skin tears is reduced. The hydrocolloid layers can be pre-connected as manufactured by removal of one half of the plow-fold release film on the upper hydrocolloid layer to adhere directly to the upper surface of the lower hydrocolloid layer of the sandwich to present to the user a single piece, integral arrangement that performs the same function as separate hydrocolloid layers but in a form that guarantees proper placement of the hydrocolloid layers and maximum adhesion to both the catheter and adhesive bonding of the sandwich. Hydrocolloid layers can be “elastic” with the same dynamic physical properties of skin thereby preventing damage to skin, dislodgment of the sandwich during respiration, patient movement or swelling of the soft tissues underlying the attachment site, as in post-trauma swelling, generalized tissue edema from kidney, lung or heart failure.

The sizes of the hydrocolloid layers 52 and 66 can vary such that the hydrocolloid layers can be provided in neonatal sizes with materials appropriate and specifically configured to meet the needs of nasogastric, orogastric, percutaneous endoscopically placed gastrostomy tube applications on premature infants, pediatric sizes and materials appropriate for securing the full range of catheters (medical tubing) not needing additional mechanical securement, adult sizes such as for securing epidural catheters and securing the full range of catheters not needing additional mechanical securement, skin protection applications, such as hydrocolloid applied to the upper ear where it joins the skull to provide protection from injury from tubing applied over the ears such as nasal cannulae or oxygen, skin tear protection for patients having at-risk skin by applying the lower hydrocolloid layer to the at-risk area to prevent tears that could be caused by shear forces generated by bed sheet transfers, movement from bed to bed and the like.

The adhesive layer arrangement shown in FIG. 1 is particularly advantageous for securing transcutaneous cannula devices, such as Huber needle and dressings, in that the bottom hydrocolloid layer is applied over the subcutaneous port to protect the skin and the upper hydrocolloid layer is applied over the Huber needle after the needle is inserted to provide stabilization and protection of placement sites. The adhesive layer arrangement can incorporate anti-microbial and bioactive compounds in the hydrocolloid formulation and, for many applications, the lower hydrocolloid layer can be translucent or clear.

In the modification of upper and lower hydrocolloid layers shown in FIG. 11, each of the upper and lower hydrocolloid layers 76 and 78 has a slot 80 therein such that the slotted hydrocolloid layers are applied at diametrically opposed approaches to a catheter which is received in the slots 80 to create a 360° seal. An additional strip of hydrocolloid 82 can be applied either before or after application of the layers 76 and 78. The adhesive layer arrangement shown in FIG. 11 is particularly advantageous for securing and sealing around chest tubes and other percutaneous drains, securing and sealing around Foley catheters and creating a colonization resistant seal, securing and sealing around feeding tubes (e.g., NG, OG, PEG), securing tubing adjacent varying height body topography utilizing the portions of hydrocolloid on either side of the slots to allow independent attachment such that one portion can be adhered to the upper lip and another portion adhered to the nose and the third layer adhered to the cheek to provide securement of an adult NG tube. Additional applications include securing and sealing around any other tube medically indicated that requires fixation, stabilization and resistance to bacterial colonization of the insertion site and/or protection of skin from bodily secretions or excretions in proximity to the insertion site or stabilization site. A tube holder 84 can be secured to the layer 76 for mechanical securement of the tubing as shown and described in U.S. patent application Ser. No. 12/620,844.

Single layers of hydrocolloid can be used to create clear window dressings with hydrocolloid borders utilizing a clear film for transparency and hydrocolloid for adherence to the skin. The use of plow-fold (U-shaped) release films with such hydrocolloid layers is particularly advantageous due to the ease of application delivering the fragile (easily wrinkled) hydrocolloid/thin film layer onto the skin application site in a flat and wrinkle free manner thus providing for an optimal occlusive seal.

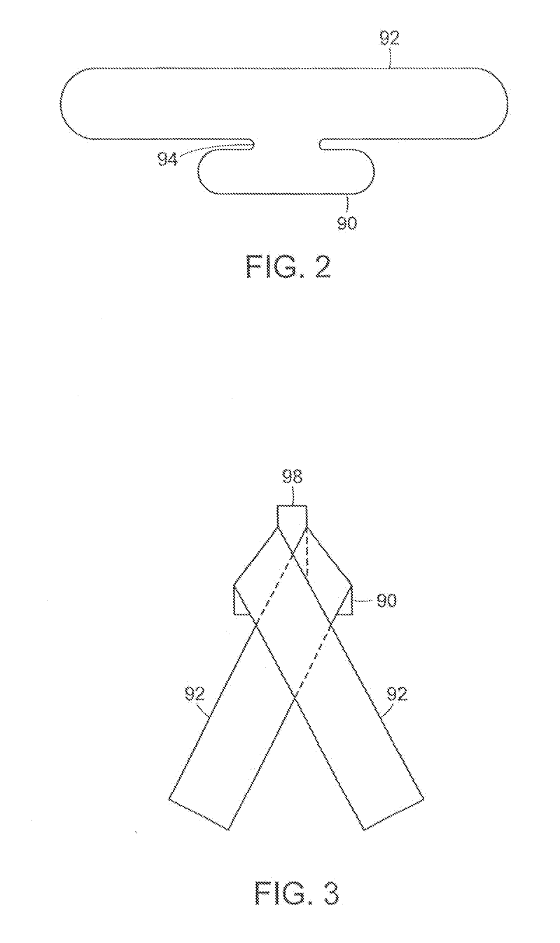

In the adhesive layer arrangement shown in FIG. 2, hydrocolloid layers have a configuration such that the lowermost (as shown) hydrocolloid layer 90 is adapted to be secured to the body, such as to the skin, and the longer uppermost (as shown) hydrocolloid layer 92 has a length to permit the layer 92, which is strip-like, to be wrapped around a catheter. A connecting portion 94 joins the layers 90 and 92 of hydrocolloid and forms a seal behind and beneath a catheter (the area between the catheter and the skin as the catheter lays parallel to the skin). The connecting portion 94 precisely aligns the hydrocolloid layer 92 with the hydrocolloid layer 90. All of the portions of the hydrocolloid layers are covered with release films, preferably the plow-fold-type previously described, and when the release film is removed the connecting portion 94 is exposed such that the connecting portion will adhere to the backside of the catheter and to the hydrocolloid strip 92 as the hydrocolloid strip is wrapped or folded around the catheter. The plow-fold release film optimizes the delivery of the hydrocolloid layers to the targeted site

As shown in FIG. 3, the hydrocolloid layer or strip 92 will cross on itself such that the crossing portions of the strip seal to each other, to a catheter 98 and to the mounting surface whether the mounting surface is skin, hydrocolloid or other material. The folded or wrapped configuration of the strip is shown in FIG. 3 with parts shaded where a seal will be formed by pinching or pressing the hydrocolloid together and against a catheter 98.

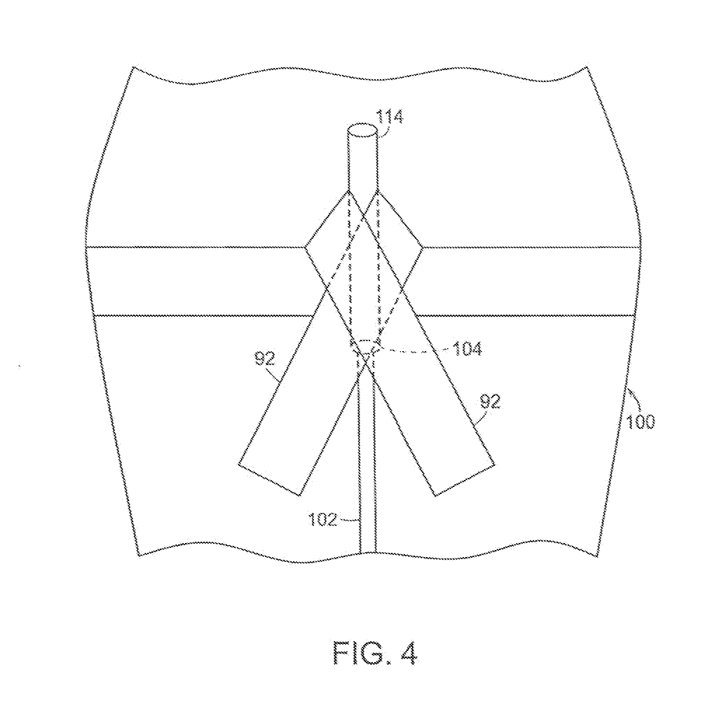

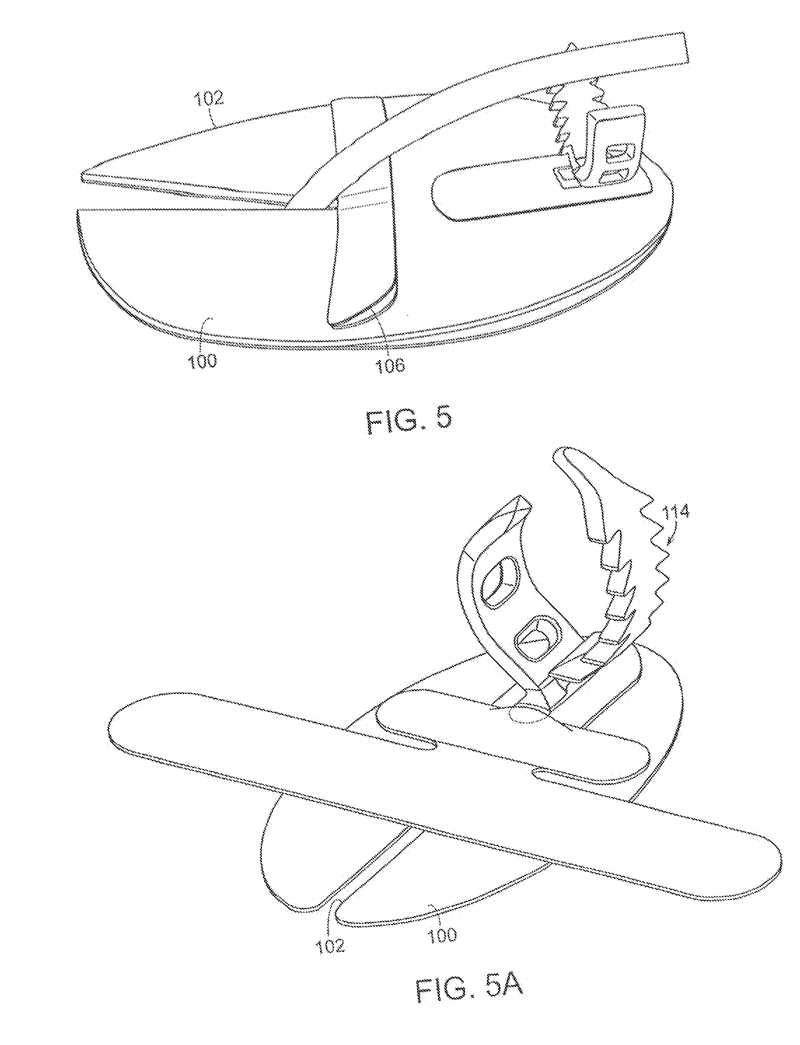

As shown in FIGS. 4-10, an adhesive layer arrangement according to the present invention includes a first layer of hydrocolloid 100 having an opening therein in the form of a slot 102 extending towards the center of the oval-shaped hydrocolloid layer 100 and terminating at a notch 104 disposed adjacent a second hydrocolloid layer 106. Hydrocolloid layer 106 extends from hydrocolloid layer 100 at a position adjacent the opening 102 and, more particularly, in alignment with notch 104; and, hydrocolloid layer 106 has an outer adherent surface 107 having a length sufficient to wrap around the catheter 112 such that the hydrocolloid layers 100 and 106 can be pressed against the catheter to create an occlusive seal at the insertion site which is aligned with notch 104. The hydrocolloid layers 100 and 106 have plow-fold release films covering adherent surfaces thereof. The method of use of the adhesive layer arrangement shown in FIGS. 4-10 involves aligning the notch 104 with an insertion site through which a catheter enters a patient's body. The release film is peeled from the layer 100. At this point, the catheter is laying along the plane of the layer 100 and the hydrocolloid layer 106 is laying against the hydrocolloid layer 100. The catheter 112 is mechanically secured in a tube holder 114, such as that disclosed in U.S. patent application Ser. No. 12/620, 844, such that the adhesive layer arrangement is essentially a two-dimensional shape. Once the release film 108 covering hydrocolloid layer 100 is removed and the hydrocolloid layer is smoothed onto the skin, the insertion site is closed with an occlusive seal by removing the release film 110 and wrapping each end of hydrocolloid layer 106 around the catheter to produce a sealing layer by adherence of the hydrocolloid layer 106 with the hydrocolloid layer 100 and with its opposite ends using the x-shape described above by grabbing the tabs of the plow-fold release film 110 and pulling towards the slot such that the hydrocolloid layer will wrap or fold around the catheter to form a seal all the way around the catheter. Accordingly, a bilaminar sealing mechanism is produced with hydrocolloid layer 106 pre-affixed to the hydrocolloid layer 100 secured to the body. The end result is a 3-dimensional cone-shaped sealing arrangement that produces an occlusive seal around the catheter at the insertion site.

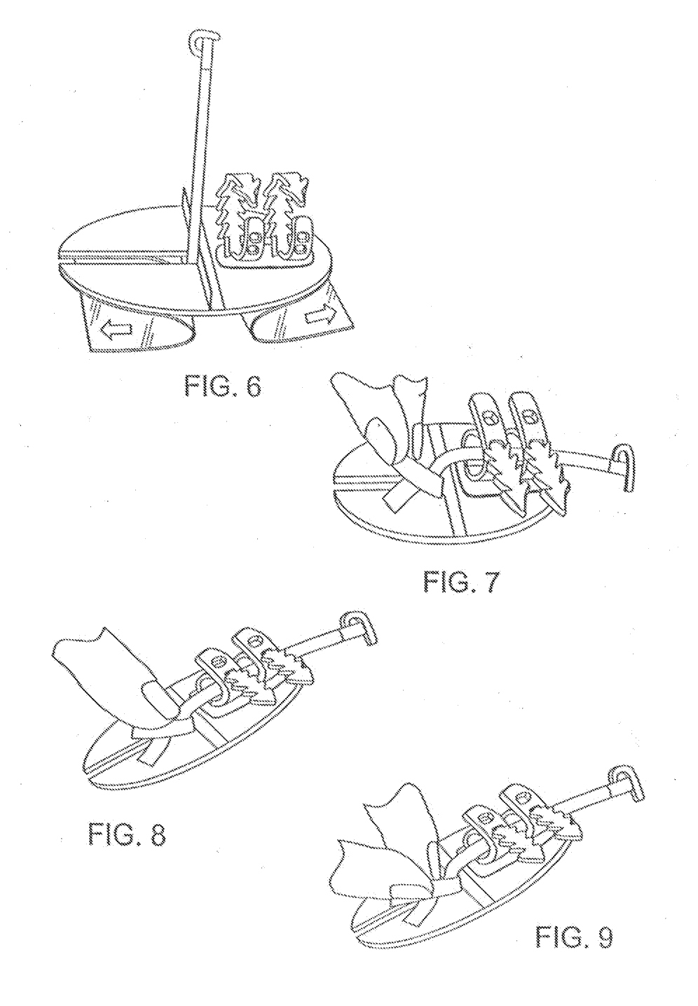

FIGS. 6, 7, 8 and 9 show the steps for use of the adhesive layer arrangement of FIGS. 4, 5, 5A and 10 with FIG. 6 showing the catheter being received in the slot or opening and in the notch to be aligned with the insertion site for the catheter. Thereafter, as shown by the arrows in FIG. 6, the plow-fold release films are removed to deliver the layer of hydrocolloid 100 to the body of the patient in a smooth and wrinkle free manner at the desired application site with the catheter in proper alignment. After the hydrocolloid layer 100 is secured, the catheter is mechanically grasped by the tube holder as shown in FIG. 7. With the adhesive layer arrangement in this condition, the opposing strips or sides of the hydrocolloid layer 106 are deployed by removing the plow-fold release films therefrom and pulling the opposing sides to form the X-shaped configuration previously described. Thereafter, as shown in FIGS. 8 and 9, the opposing sides of the hydrocolloid layer 106 are pressed together, FIG. 8, and pinched, FIG. 9, to form the occlusive seal at the insertion site by completely engaging the catheter and the insertion site.

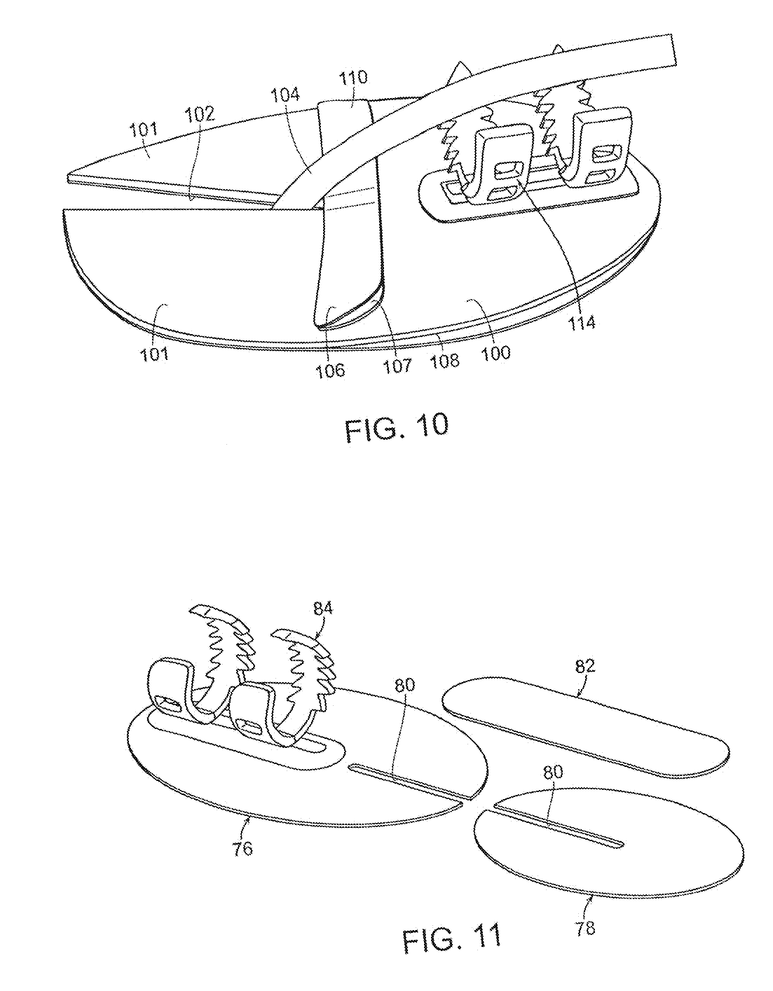

The slot 102 incorporates three separate components of the release film/layer. The three separate release layer components prevent the release layer from tearing on application due to impact with the catheter 98 when notch 104 is positioned in close proximity to the catheter 98. Notch 104 locates the base layer in close proximity to the insertion site to protect and adhere to the skin and allows placement of the hydrocolloid in close proximity to the catheter 98. The three piece release film component assembly allows each portion of the adhesive base layer 100 to apply in a smooth wrinkle free manner to the desired application site without having to move the base layer once the base layer is placed in a desired location. In addition, the release film is able to release smoothly from the adhesive layer due to the independent release action of the three release film components as shown in FIG. 10, one component from each limb (portion) 101 of the hydrocolloid located lateral to the central slot 102 of the hydrocolloid base layer 100 and one release film component from the remaining portion of the lower adhesive surface 108 of the hydrocolloid base layer 100 thereby applying to the application site in a flat and wrinkle free manner. The three separate release film components are configured to ensure no tearing of the release film occurs or residual film is left behind. This is done by ensuring the release film fold point (the point where the release film folds back on itself and becomes the proximal portion of the release film contiguous pull tab) is adjacent the end of the ovoid notch 104. When configured correctly, the release film pulls from beneath the device without residue, tearing or disruption of the release film and provides a flat and wrinkle free application of adhesive base layer 100 to the chosen application site. The multiple component release film assembly ensures the adhesive base layer 100 contributes to the occlusive seal formation surrounding the catheter insertion site. The notch 104 in a release film is advantageous whether the release film is plow-folded or not.

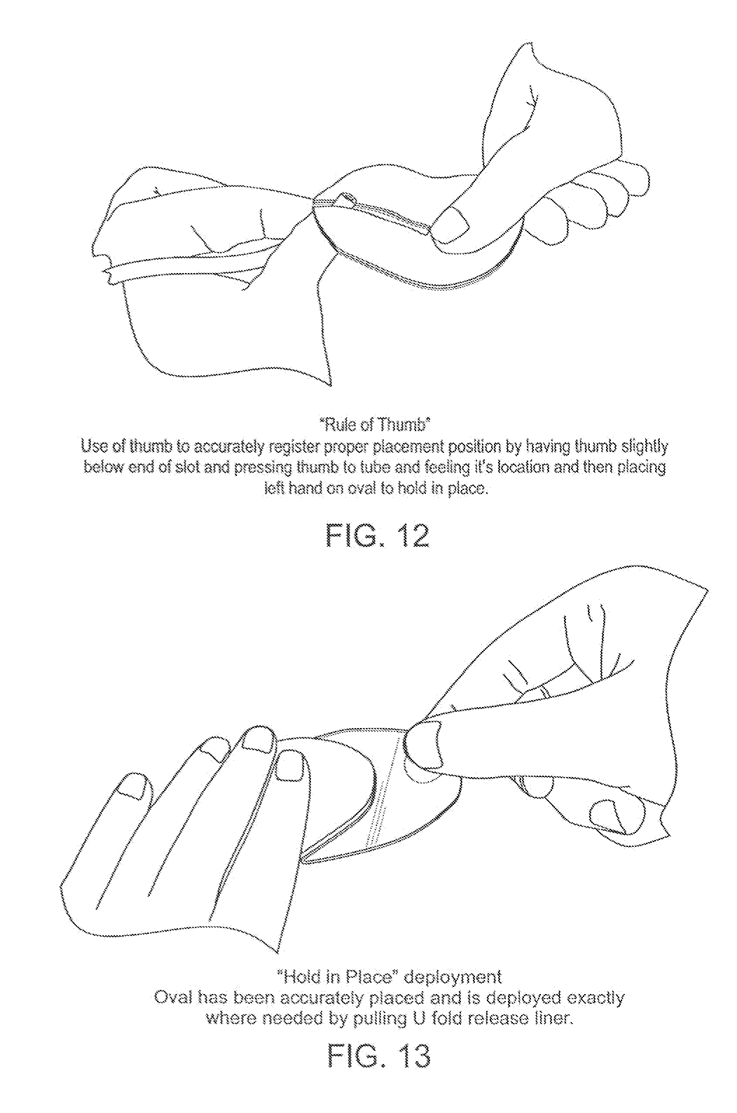

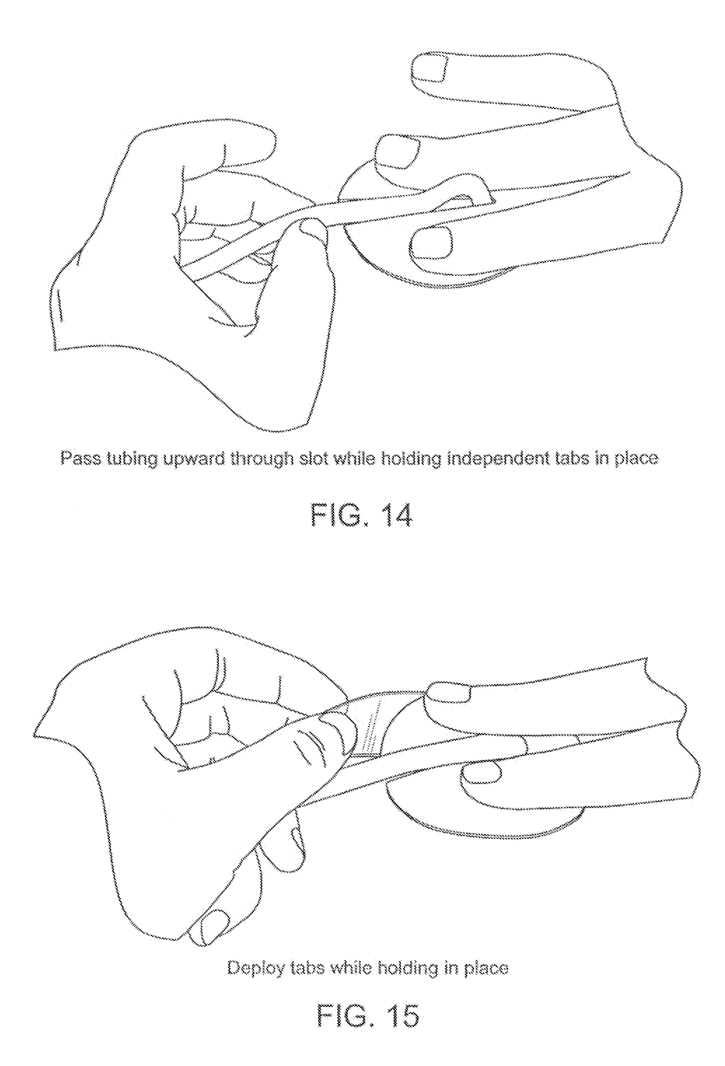

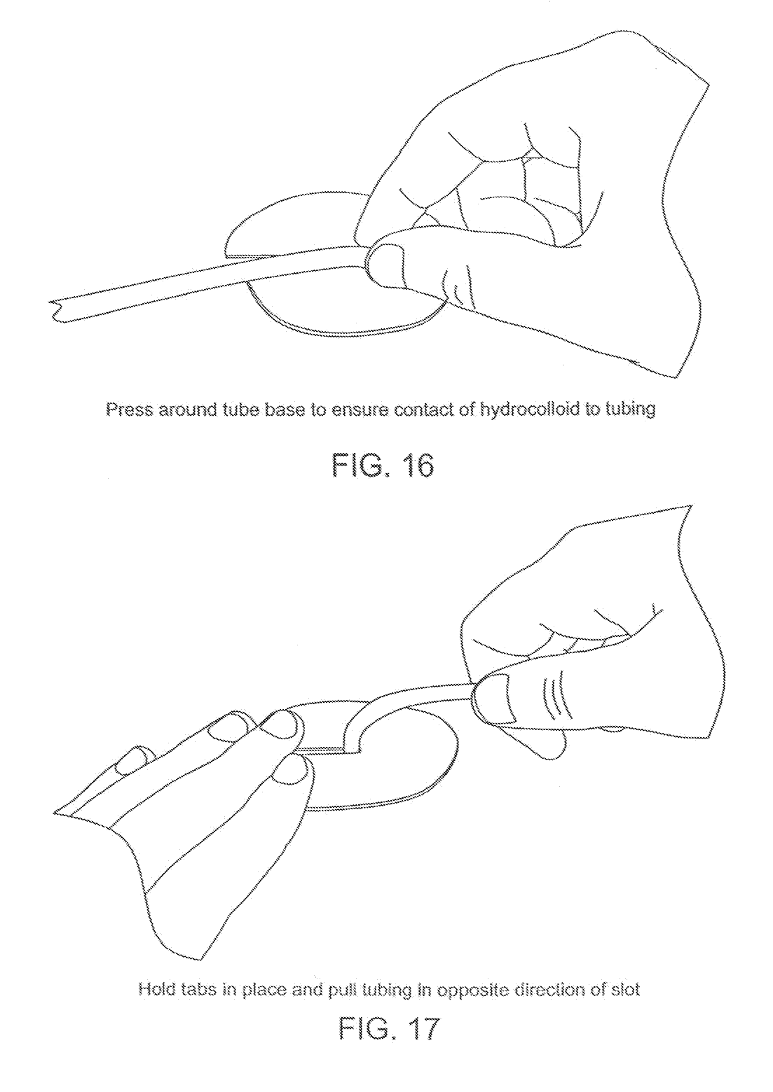

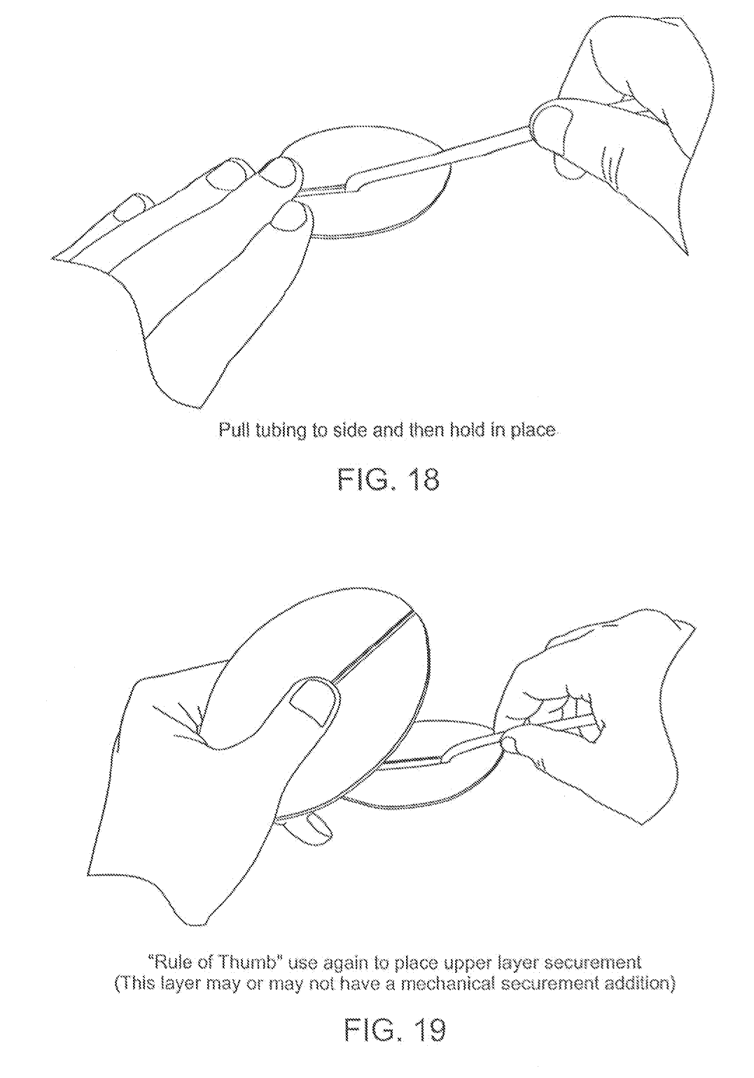

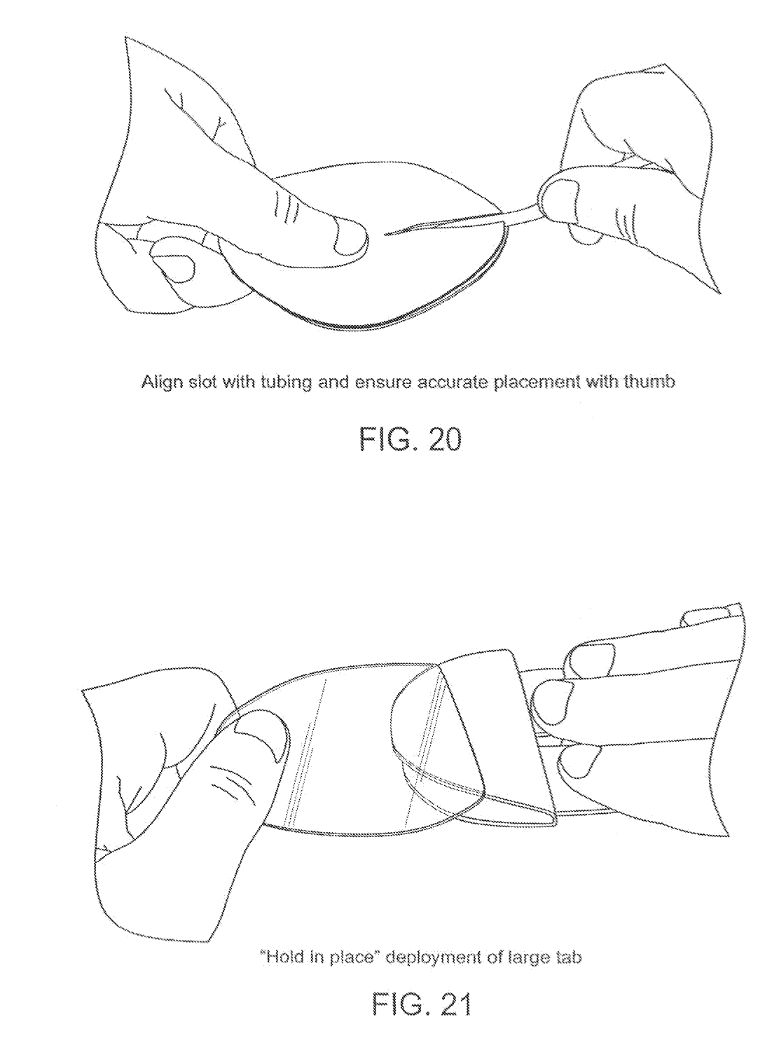

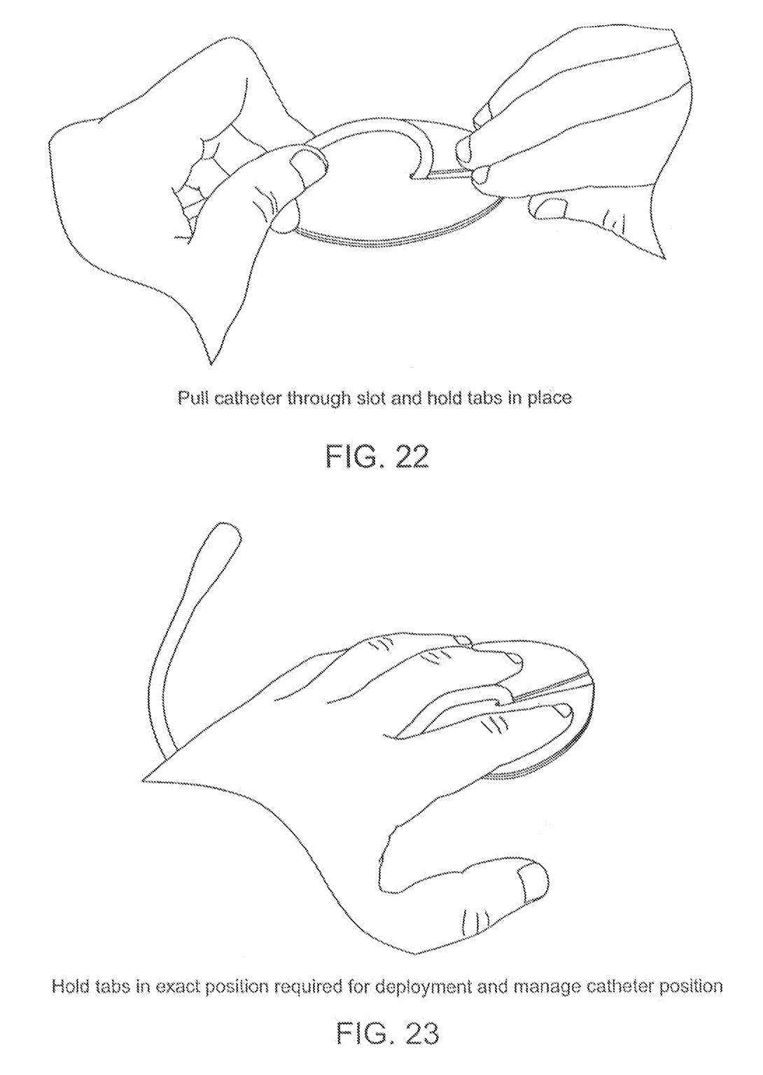

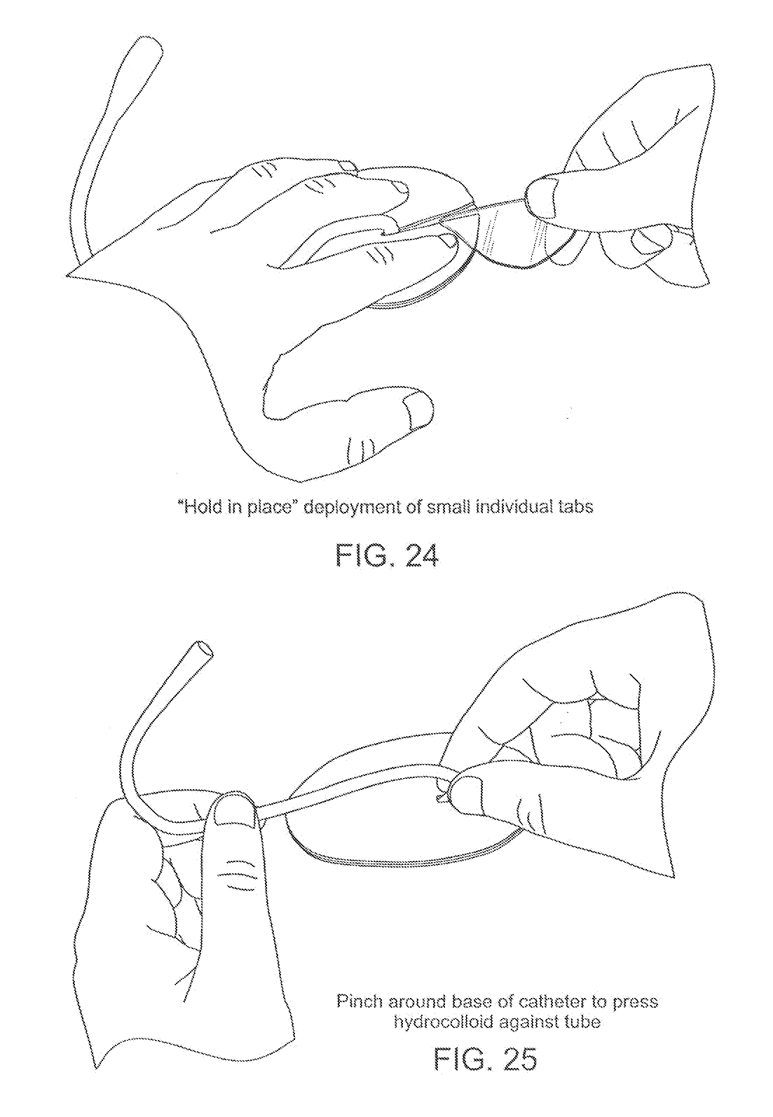

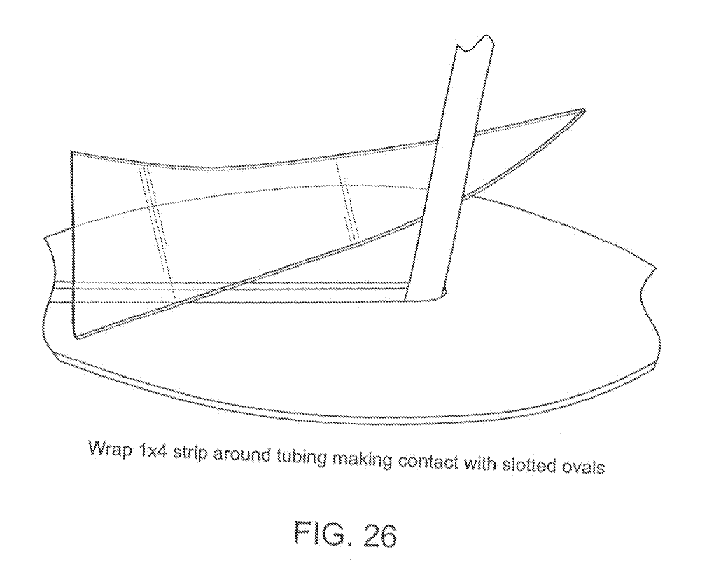

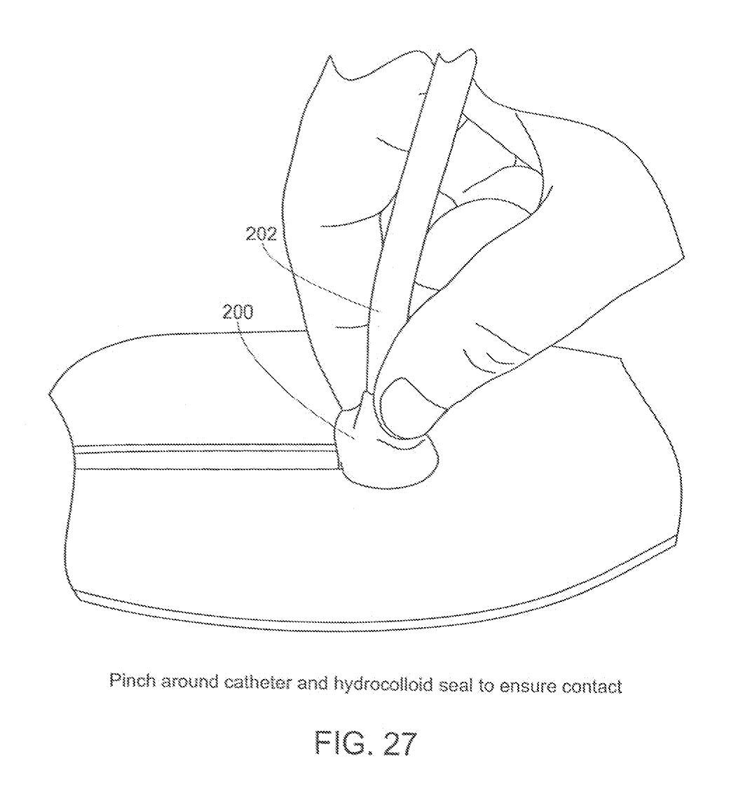

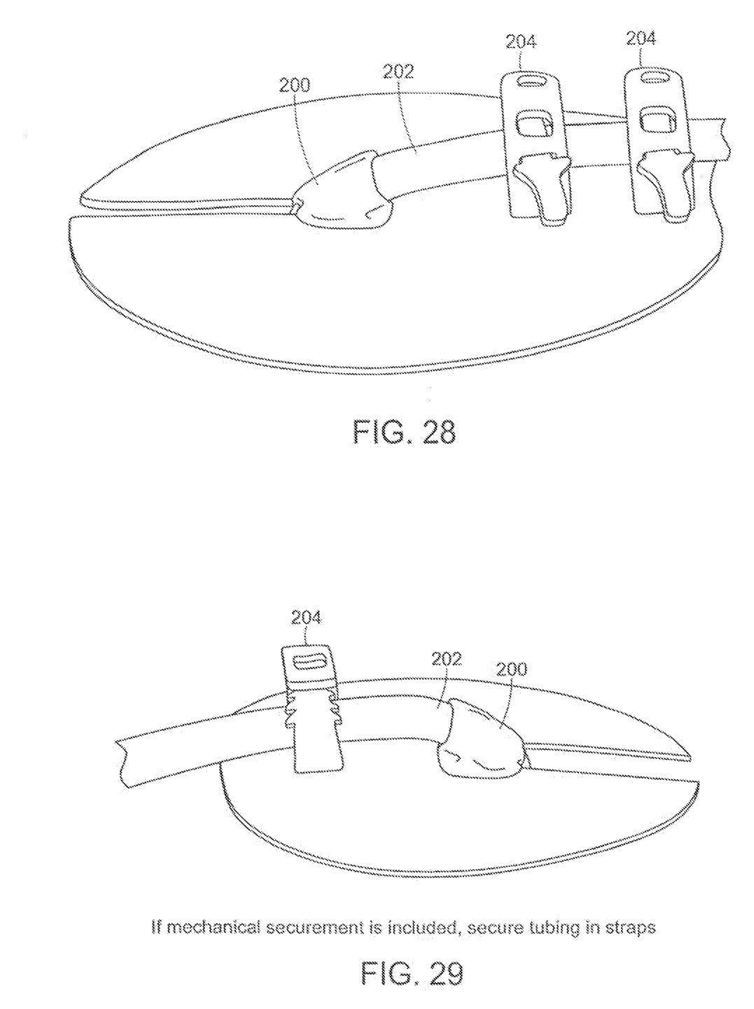

FIGS. 12-29 show method steps for applying the adhesive layer arrangement according to the present invention with captions describing the steps. FIG. 27 shows the pinching of the hydrocolloid at 200 to create a seal with a catheter 202. FIG. 28 shows the pinched seal 200 sealing the catheter 202 which is then held by straps 204 to provide mechanical securement of the catheter which is also shown in FIG. 29 using a single set of straps 204.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. An adhesive layer arrangement for securing catheters to patients comprising
a lower layer of hydrocolloid having first and second opposing ends, a bottom adherent surface extending between said first and second opposing ends for adhering to the body of a patient and a top surface extending between said first and second opposing ends;
a plow-fold release film covering said bottom adherent surface of said lower layer of hydrocolloid having a tab extending away from said bottom adherent surface; and
an upper layer of hydrocolloid having a bottom adherent surface for adhering to said top surface of said lower layer of hydrocolloid and to the catheter whereby, when said upper layer of hydrocolloid is pressed against said lower layer of hydrocolloid with the catheter disposed on said top surface of said lower layer of hydrocolloid, said upper layer of hydrocolloid deforms to securely grip the catheter and, in combination with said lower layer of hydrocolloid, secures the catheter to the patient's body preventing rotational, longitudinal and peeling movement of the catheter.

2. An adhesive layer arrangement for securing catheters to patients as recited in claim 1 wherein said lower and upper layers of hydrocolloid have oval shapes and are separate layers.

3. An adhesive layer arrangement for securing catheters to patients as recited in claim 2 wherein a polyurethane film is disposed on said top surface of said lower layer of hydrocolloid.

4. An adhesive layer arrangement for securing catheters to patients as recited in claim 2 wherein said top surface of said lower layer of hydrocolloid is adherent to comingle with said upper layer of hydrocolloid.

5. An adhesive layer arrangement for securing a catheter inserted into the body of a patient and for creating an occlusive seal at the insertion site at which the catheter enters the patient's body comprising
a first layer of hydrocolloid having a bottom adherent surface for adhering to the body of a patient and an opening for receiving the catheter;
a plow-fold release film covering said bottom adherent surface of said first layer of hydrocolloid having a tab extending away from said bottom adherent surface; and
a second layer of hydrocolloid extending from said first layer of hydrocolloid at a position adjacent said opening and having an outer adherent surface with a length sufficient to wrap around the catheter whereby said first and second layers of hydrocolloid can be pressed against the catheter and the body of the patient to deform and create an occlusive seal at the insertion site.

6. An adhesive layer arrangement for securing a catheter inserted into the body of a patient and for creating an occlusive seal at the insertion site at which the catheter enters the patient's body as recited in claim 5 wherein said opening is a slot in said first layer of hydrocolloid.

7. An adhesive layer arrangement for securing a catheter inserted into the body of a patient and for creating an occlusive seal at the insertion site at which the catheter enters the patient's body as recited in claim 6 wherein said first layer of hydrocolloid has an oval shape with opposing ends and said slot extends from one of said ends to terminate in a notch adapted to be aligned with the insertion site and to receive the catheter.

8. An adhesive layer arrangement for securing a catheter inserted into the body of a patient and for creating an occlusive seal at the insertion site at which the catheter enters the patient's body as recited in claim 5 wherein said first and second layers of hydrocolloid are integrally formed.

9. An adhesive layer arrangement for securing a catheter inserted into the body of a patient and for creating an occlusive seal at the insertion site at which the catheter enters the patient's body as recited in claim 5 and further comprising opposing plow-fold release films covering said outer adherent surface of said second layer of hydrocolloid having tabs whereby pulling said tabs exposes said outer adherent surface and wraps said second layer of hydrocolloid around the catheter.
